(19) United States
(12) Patent Application Publication
(10) Pub. No.: US 2025/0241933 A1
(43) Pub. Date: Jul. 31, 2025

(54) METHODS OF TREATING SLEEP APNEA WITH A COMBINATION OF A CANNABINOID AND A CARBONIC ANHYDRASE INHIBITOR

(71) Applicant: APNIMED, INC. (DELAWARE), Cambridge, MA (US)

(72) Inventors: Luigi TARANTO-MONTEMURRO, Brookline, MA (US); Ronald FARKAS, Clarksville, MD (US); David P. WHITE, Denver, CO (US); Lawrence G. MILLER, Cambridge, MA (US)

(21) Appl. No.: 18/876,682

(22) PCT Filed: Jun. 20, 2023
(86) PCT No.: PCT/US2023/025698

§ 371 Date: Dec. 19, 2024

Related U.S. Application Data
(60) Provisional application No. 63/353,903, filed on Jun. 21, 2022.

(51) Int. Cl. A61K 31/00; A61K 31/18; A61K 31/36; A61K 31/423; A61K 31/428; A61K 31/54; A61P 11/04

(52) U.S. Cl. CPC A61K 31/658; A61K 31/18; A61K 31/36; A61K 31/423; A61K 31/428; A61K 31/54; A61P 11/04

(57) ABSTRACT

Methods of treating sleep apnea and snoring comprising administering a combination of a cannabinoid and a carbonic anhydrase inhibitor (CAI), and optionally a muscarinic receptor antagonist (MRA), are described herein. Pharmaceutical compositions comprising a cannabinoid and a carbonic anhydrase inhibitor (CAI), and optionally a MRA, are also described. In some embodiments, the cannabinoid is a non-THC cannabinoid. In some embodiments, the CAI is selected from the group consisting of dichlorophenamide, zonisamide, ethoxzolamide, topiramate, and sultiame, or a pharmaceutically acceptable salt thereof

### CROSS-REFERENCE TO RELATED APPLICATION

This application claims the benefit of and priority to U.S. provisional application No. 63/353,903, filed Jun. 21, 2022, the entire contents of which are incorporated herein by reference.

### TECHNICAL FIELD

The present invention provides methods of treating sleep apnea and snoring comprising administering a cannabinoid and a carbonic anhydrase inhibitor (CAI).

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of (i) a cannabinoid and (ii) a carbonic anhydrase inhibitor (CAI) selected from the group consisting of dichlorophenamide, zonisamide, ethoxzolamide, topiramate, and sultiame, or a pharmaceutically acceptable salt thereof, in the absence of a norepinephrine reuptake inhibitor (NRI) therapy.

Embodiments of this aspect of the invention may include one or more of the following optional features. In some embodiments, the (i) cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA), or any combination thereof, or a pharmaceutically acceptable salt thereof. In some embodiments, the cannabinoid is CBD. In some embodiments, the cannabinoid is THC. In some embodiments, the (i) cannabinoid is dronabinol. In some embodiments, the CAI is sultiame, or a pharmaceutically acceptable salt thereof. In some embodiments, the sultiame or a pharmaceutically acceptable salt thereof, is administered at a dosage of from about 200 to about 400 mg. In some embodiments, CBD is administered at a dosage of from about 0.5 to about 300 mg. In some embodiments, THC is administered at a dosage of from about 0.1 to about 30 mg. In some embodiments, the method further comprises administering (iii) a muscarinic receptor antagonist (MRA) to the subject. In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the MRA is (R)-oxybutynin or a pharmaceutically acceptable salt thereof. In some embodiments, the oxybutynin or pharmaceutically acceptable salt thereof is administered at a dose of from about 1 to about 15 mg. In some embodiments, the oxybutynin or pharmaceutically acceptable salt thereof is administered at a dose of from about 2 mg to about 10 mg. In some embodiments, the (R)-oxybutynin or pharmaceutically acceptable salt thereof is administered at a dose of from about 0.5 to about 10 mg. In some embodiments, the (R)-oxybutynin or pharmaceutically acceptable salt thereof is administered at a dose of from about 1 mg to about 5 mg. In some embodiments, the (i) cannabinoid and (ii) CAI are administered in single composition. In some embodiments, the (i) cannabinoid, (ii) CAI, and (iii) MRA are administered in single composition. In some embodiments, the single composition is an oral administration form. In some embodiments, the oral administration form is a syrup, pill, tablet, troche, capsule, or patch. In some embodiments, the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, wherein the condition associated with pharyngeal airway collapse is simple snoring. In some embodiments, the subject is in a non-fully conscious state. In some embodiments, the non-fully conscious state is sleep.

Another aspect of the invention provides a method of treating a subject having a condition associated with pharyngeal airway collapse, the method comprising administering to a subject in need thereof an effective amount of (i) a non-tetrahydrocannabinol (non-THC) cannabinoid and (ii) a carbonic anhydrase inhibitor (CAI), or a pharmaceutically acceptable salt thereof, in the absence of a norepinephrine reuptake inhibitor (NRI) therapy.

Embodiments of this aspect of the invention may include one or more of the following optional features. In some embodiments, the (i) non-THC cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), and cannabicyclol (CBL), or any combination thereof, or a pharmaceutically acceptable salt thereof. In some embodiments, the non-THC cannabinoid is CBD. In some embodiments, the (ii) CAI is selected from the group consisting of acetazolamide, dichlorophenamide, dorzolamide, brinzolamide, methazolamide, zonisamide, ethoxzolamide, topiramate, sultiame, or a pharmaceutically acceptable salt thereof. In some embodiments, the CAI is acetazolamide or a pharmaceutically acceptable salt thereof. In some embodiments, the CAI, such as acetazolamide, or a pharmaceutically acceptable salt thereof, is administered at a dosage of from about 250 mg to about 750 mg. In some embodiments, the CAI, such as acetazolamide, or a pharmaceutically acceptable salt thereof, is administered at a dosage of about 500 mg. In some embodiments, CBD is administered at a dosage of from about 0.5 to about 300 mg. In some embodiments, the method further comprises administering (iii) a muscarinic receptor antagonist (MRA) to the subject. In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the MRA is (R)-oxybutynin or a pharmaceutically acceptable salt thereof. In some embodiments, the oxybutynin or pharmaceutically acceptable salt thereof is administered at a dose of from about 1 to about 15 mg. In some embodiments, the oxybutynin or pharmaceutically acceptable salt thereof is administered at a dose of from about 2 mg to about 10 mg. In some embodiments, the (R)-oxybutynin or pharmaceutically acceptable salt thereof is administered at a dose of from about 0.5 to about 10 mg. In some embodiments, the (R)-oxybutynin or pharmaceutically acceptable salt thereof is administered at a dose of from about 1 mg to about 5 mg. In some embodiments, the (i) non-THC cannabinoid and (ii) CAI are administered in single composition. In some embodiments, the (i) non-THC cannabinoid, (ii) CAI, and (iii) MRA are administered in single composition. In some embodiments, the single composition is an oral administration form. In some embodiments, the oral administration form is a syrup, pill, tablet, troche, capsule, or patch. In some embodiments, the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep.

Another aspect of the present invention provides a pharmaceutical composition comprising (i) a cannabinoid and (ii) a carbonic anhydrase inhibitor (CAI) selected from the group consisting of dichlorophenamide, zonisamide, ethoxzolamide, topiramate, and sultiame, or a pharmaceutically acceptable salt thereof, and one or more pharmaceutically acceptable carriers or excipients, wherein the composition does not contain a norepinephrine reuptake inhibitor (NRI).

Embodiments of this aspect of the invention may include one or more of the following optional features. In some embodiments, the (i) cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA), or any combination thereof, or a pharmaceutically acceptable salt thereof. In some embodiments, the cannabinoid is CBD. In some embodiments, the cannabinoid is THC. In some embodiments, the (i) cannabinoid is dronabinol. In some embodiments, the CAI is sultiame, or a pharmaceutically acceptable salt thereof. In some embodiments, the sultiame, or a pharmaceutically acceptable salt thereof, is present in an amount of from about 200 to about 400 mg. In some embodiments, CBD is present in an amount of from about 0.5 to about 300 mg. In some embodiments, THC is present in an amount of from about 0.1 to about 30 mg.

Another aspect of the present invention provides a pharmaceutical composition comprising (i) a non-tetrahydrocannabinol (non-THC) cannabinoid and (ii) a carbonic anhydrase inhibitor (CAI), or a pharmaceutically acceptable salt thereof, and one or more pharmaceutically acceptable carriers or excipients, wherein the composition does not contain a norepinephrine reuptake inhibitor (NRI).

Embodiments of this aspect of the invention may include one or more of the following optional features. In some embodiments, the (i) non-THC cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), and cannabicyclol (CBL), or any combination thereof, or a pharmaceutically acceptable salt thereof. In some embodiments, the non-THC cannabinoid is CBD. In some embodiments, the (ii) CAI is selected from the group consisting of acetazolamide, dichlorophenamide, dorzolamide, brinzolamide, methazolamide, zonisamide, ethoxzolamide, topiramate, sultiame, or a pharmaceutically acceptable salt thereof. In some embodiments, the CAI is acetazolamide or a pharmaceutically acceptable salt thereof. In some embodiments, the CAI, such as acetazolamide, or a pharmaceutically acceptable salt thereof, is present in an amount of from about 250 mg to about 750 mg. In some embodiments, the CAI, such as acetazolamide, or a pharmaceutically acceptable salt thereof, is present in an amount of about 500 mg. In some embodiments, CBD is present in an amount of from about 0.5 to about 300 mg. In some embodiments, the composition further comprises (iii) a muscarinic receptor antagonist (MRA). In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the MRA is (R)-oxybutynin or a pharmaceutically acceptable salt thereof. In some embodiments, the oxybutynin or a pharmaceutically acceptable salt thereof is present in an amount of from about 1 to about 15 mg. In some embodiments, the oxybutynin or a pharmaceutically acceptable salt thereof is present in an amount of from about 2 mg to about 10 mg. In some embodiments, the (R)-oxybutynin or a pharmaceutically acceptable salt thereof is present in an amount of from about 0.5 to about 10 mg. In some embodiments, the (R)-oxybutynin or a pharmaceutically acceptable salt thereof is present in an amount of from about 1 mg to about 5 mg. In some embodiments, the (i) cannabinoid, (ii) CAI, and, optionally the (iii) MRA, are formulated in a single composition. In some embodiments, the (i) non-THC cannabinoid, (ii) CAI, and, optionally the (iii) MRA, are formulated in a single composition. In some embodiments, the single composition is an oral administration form. In some embodiments, the oral administration form is a syrup, pill, tablet, troche, capsule, or patch.

In some embodiments, the pharmaceutical compositions are for use in treating a subject having a condition associated with pharyngeal airway collapse. In some embodiments, the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep.

Another aspect of the present invention provides a kit comprising (i) a cannabinoid and (ii) a carbonic anhydrase inhibitor (CAI) selected from the group consisting of dichlorophenamide, zonisamide, ethoxzolamide, topiramate, and sultiame, or a pharmaceutically acceptable salt thereof, and optionally (iii) a muscarinic receptor antagonist (MRA), wherein the kit does not contain a norepinephrine reuptake inhibitor (NRI).

Another aspect of the present invention provides a kit comprising (i) a non-THC cannabinoid and (ii) a carbonic anhydrase inhibitor (CAI) selected, or a pharmaceutically acceptable salt thereof, and optionally (iii) a muscarinic receptor antagonist (MRA), wherein the kit does not contain a norepinephrine reuptake inhibitor (NRI). In some embodiments, the kits are for use in treating a subject having a condition associated with pharyngeal airway collapse.

Another aspect of the present invention provides a cannabinoid and a carbonic anhydrase inhibitor (CAI) selected from the group consisting of dichlorophenamide, zonisamide, ethoxzolamide, topiramate, and sultiame, or a pharmaceutically acceptable salt thereof, and optionally a muscarinic receptor antagonist (MRA), for use in treating a subject having a condition associated with pharyngeal airway collapse in the absence of a norepinephrine reuptake inhibitor (NRI) therapy.

Another aspect of the present invention provides a non-THC cannabinoid and a carbonic anhydrase inhibitor (CAI), or a pharmaceutically acceptable salt thereof, and optionally a muscarinic receptor antagonist (MRA), for use in treating a subject having a condition associated with pharyngeal airway collapse in the absence of a norepinephrine reuptake inhibitor (NRI) therapy. Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The following figures are provided by way of example and are not intended to limit the scope of the claimed invention.

FIG. 1 is a graphic illustration of an obstructive apnea. The top channel shows the electroencephalogram (EEG) pattern of sleep. The next channel represents airflow. The next three channels show ventilator effort by movements of the rib cage and abdomen and changes in esophageal pressure, all of which reflect a respiratory effort against an occluded upper airway. The last channel indicates oxyhemoglobin saturation.

### DETAILED DESCRIPTION

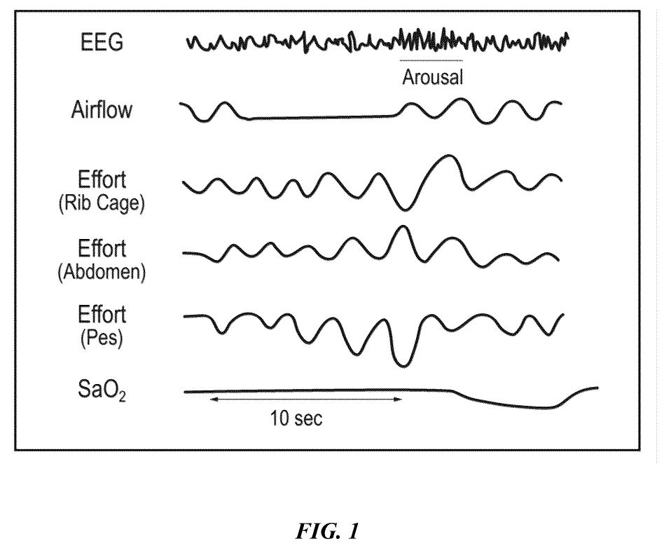

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualisable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruehland et al., The new AASM criteria for scoring hypopneas: Impact on the apnea hypopnea index. SLEEP 2009; 32(2): 150-157).

FIG. 1 is a graphic illustration of an obstructive apnea. The top channel shows the electroencephalogram (EEG) pattern of sleep. The next channel represents airflow. The next three channels show ventilatory effort by movements of the rib cage and abdomen and changes in esophageal pressure, all of which reflect a respiratory effort against an occluded upper airway. The last channel indicates oxyhemoglobin saturation.

When a stringent definition of OSA is used (an AHI of >15 events per hour or AHI>5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young et al., WMJ 2009; 108:246; Peppard et al., Am J Epidemiol 2013; 177:1006.)

The present leading treatment is continuous positive airway pressure (CPAP). CPAP is effective in virtually all patients, and approximately 85% of diagnosed patients are prescribed CPAP, but compliance is low. Patients find CPAP uncomfortable and often intolerable; at least 30% of patients (up to 80%) are regularly non-adherent and thus untreated (Weaver, Proc Am Thorac Soc. 2008 Feb. 15; 5(2): 173-178). Other treatment modalities with variable rates of success include oral appliances (10%) and surgery (5%), but neither is likely to be effective across the general population.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. See, e.g., Proia and Hudgel, Chest. 1991 August; 100(2):416-21; Brownell et al., N Engl J Med 1982, 307:1037-1042; Sangal et al., Sleep Med. 2008 July; 9(5):506-10. Epub 2007 Sep. 27; Marshall et al. p. 2008 June; 31(6):824-31; Eckert et al., Clin Sci (Lond). 2011 June; 120(12); 505-14; Taranto-Montemurro et al., Sleep. 2017 Feb. 1; 40(2).

In a recent study, a combination of atomoxetine and oxybutynin, referred to as “ato-oxy,” administered before bedtime has been shown to reduce OSA in patients with a wide range of severity. The ato-oxy combination, which was administered for one night, reduced the number of obstructive events, improved the overnight oxygen desaturation, and enhanced the genioglossus muscle activity in a group of unselected patients with OSA. The data collected in the proof-of-concept trial showed that it was possible to improve or abolish OSA using drugs with specific neurotransmitter profiles administered systemically. See Taranto-Montemurro, L. et al., The Combination of Atomoxetine and Oxybutynin Greatly Reduces Obstructive Sleep Apnea Severity. A Randomized, Placebo-controlled, Double-Blind Crossover Trial. Am J Respir Crit Care Med 2019 May 15; 199(10): 1267-1276.

There remains a need for further therapies for treating conditions associated with pharyngeal airway collapse such as sleep apnea.

### Methods of Treatment

The methods described herein include methods for the treatment of disorders associated with pharyngeal airway muscle collapse during sleep. In some embodiments, the disorder is sleep apnea (e.g., obstructive sleep apnea (OSA)) or snoring (e.g., simple snoring). Generally, the methods include administering an effective amount of (i) a cannabinoid and (ii) a carbonic anhydrase inhibitor (CAI) to a subject who is in need of, or who has been determined to be in need of, such treatment, in the absence of a norepinephrine reuptake inhibitor (NRI) therapy. In other embodiments, the methods further administer a muscarinic receptor antagonist (MRA).

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA may result in decreased AHI. Measurement of OSA disease and symptoms may be, for example, by polysomnography (PSG).

In general, an “effective amount” of a compound refers to an amount sufficient to elicit the desired biological response, e.g., to treat a condition associated with pharyngeal airway collapse, e.g., to treat sleep apnea or snoring. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

An effective amount can be administered in one or more administrations, applications or dosages. The compositions can be administered from one or more times per day to one or more times per week; including once every other day. In some embodiments, the compositions are administered daily. In some embodiments, the compositions are administered daily before sleep time, e.g., immediately before sleep time or 15-60 minutes before sleep time. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, a “norepinephrine reuptake inhibitor (NRI) therapy” refers to the administration of a norepinephrine reuptake inhibitor (NRI). Norepinephrine reuptake inhibitors (NRIs) include, but are not limited to, the selective NRIs, e.g., amedalin (UK-3540-1), atomoxetine (Strattera), CP-39,332, daledalin (UK-3557-15), edivoxetine (LY-2216684), esreboxetine, lortalamine (LM-1404), nisoxetine (LY-94,939), reboxetine (Edronax, Vestra), talopram (Lu 3-010), talsupram (Lu 5-005), tandamine (AY-23,946), viloxazine (Vivalan); and the non-selective NRIs, e.g., amitriptiline, amoxapine, bupropion, ciclazindol, desipramine, desvenlafaxine, dexmethilphenidate, diethylpropion, doxepin, duloxetine, imipramine, levomilnacipran, manifaxine (GW-320,659), maprotiline, methylphenidate, milnacipran, nefazodone, nortriptyline, phendimetrazine, phenmetrazine, protryptyline, radafaxine (GW-353,162), tapentadol (Nucynta), teniloxazine (Lucelan, Metatone) and venlafaxine; and pharmaceutically acceptable salts thereof. Subjects receiving treatment according to the present disclosure in the absence of a NRI therapy do not receive administration of a norepinephrine reuptake inhibitor.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, Berge, S M. et al., “Pharmaceutical Salts,” J. Pharm. Sci., 1977; 66:1-19 which is incorporated herein by reference.)

As used herein, the term “unit dosage form” is defined to refer to the form in which the compound is administered to a subject. Specifically, the unit dosage form can be, for example, a pill, capsule, or tablet. In some embodiments, the unit dosage form is a capsule.

As used herein, “solid dosage form” means a pharmaceutical dose(s) in solid form, e.g. tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

Oxybutynin is the generic name for the pharmaceutical substance with the chemical name 4-diethylamino-2-butynylphenylcyclohexylglycolate or 4-(diethylamino) but-2-ynyl 2-cyclohexyl-2-hydroxy-2-phenylacetate, and its pharmaceutically acceptable salts. In various embodiments, oxybutynin may be a racemic mixture of R-and S-enantiomers, or an isolated enantiomer, e.g., the R-enantiomer. In various embodiments, oxybutynin may be oxybutynin chloride or (R)-oxybutynin chloride.

Acetazolamide is the generic name of the pharmaceutical substance with the chemical name N-(5-Sulfamoyl-1,3,4-thiadiazol-2-yl) acetamide, and its pharmaceutical salts. Acetazolamide is available as a generic medication as well as sold under the trade names Diamox, Dacarb, and others.

“Cannabinoids” are a group of compounds including the endocannabinoids, the phytocannabinoids and those which are neither endocannabinoids nor phytocannabinoids, hereinafter “syntho-cannabinoids”. “Endocannabinoids” are endogenous cannabinoids, which are high affinity ligands of CB1 and CB2 receptors. “Phytocannabinoids” are cannabinoids that originate in nature and can be found in the cannabis plant. The phytocannabinoids can be present in an extract including a botanical drug substance, isolated, or reproduced synthetically. “Syntho-cannabinoids” are those compounds capable of interacting with the cannabinoid receptors (CB1 and/or CB2) but are not found endogenously or in the cannabis plant.

“Non-THC cannabinoids” are a cannabinoids described herein that are not THC (delta-9-tetrahydrocannabinol (49-THC)) or any of its homologs or isomers (e.g., THC, dronabinol, CBN, CBO, D8-THC, trans-A10-THC, cis-A10-THC, THCV, A8-THCV, or A9-THCV). “Non-TCH cannabinoids” can also be cannabinoids without delta-9-tetrahydrocannabinols. In some embodiments, the non-THC cannabinoid is cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), and cannabicyclol (CBL), or a pharmaceutically acceptable salt thereof.

In some embodiments, the methods include administering a dose of from about 0.5 to about 300 mg of CBD (e.g., daily). In some embodiments, the dose of CBD or a pharmaceutically acceptable salt thereof is from about 1 to about 100 mg. In some embodiments, the dose of CBD or a pharmaceutically acceptable salt thereof is from about 1 mg to about 10 mg. In some embodiments, the dose of CBD or a pharmaceutically acceptable salt thereof is from about 10 mg to about 100 mg. In some embodiments, the CBD is administered orally. In some embodiments, the CBD is administered sublingually.

In some embodiments, the methods include administering a dose of from about 100 mg to about 500 mg of sultiame, or a pharmaceutically acceptable salt thereof (e.g., daily). In some embodiments, the dose of sultiame or a pharmaceutically acceptable salt thereof is from about 200 mg to about 400 mg. In some embodiments, the sultiame is administered orally.

In some embodiments, the methods include administering a dose of from about 0.1 to about 30 mg of THC. In some embodiments, the methods include administering a dose of from about 1 to about 20 mg of THC. In some embodiments, the dose of THC or a pharmaceutically acceptable salt thereof is from about 0.5 to about 20 mg. In some embodiments, the dose of THC or a pharmaceutically acceptable salt thereof is from about 0.25 to about 10 mg. In some embodiments, the THC is administered orally. In some embodiments, the THC is administered sublingually.

In some embodiments, the methods include administering a dose of from about 1 mg to about 20 mg of dronabinol (e.g., daily). In some embodiments, the methods include administering a dose of from about 2.5 mg to about 10 mg of dronabinol (e.g., daily). In some embodiments, the methods include administering a dose of from about 5 mg to about 10 mg of dronabinol (e.g., daily). In some embodiments, the methods include administering a dose of about 5 mg of dronabinol (e.g., daily). In some embodiments, the methods include administering a dose of about 10 mg of dronabinol (e.g., daily).

In some embodiments, the methods include administering a dose of from about 0.25 mg to about 20 mg of nabilone (e.g., from about 0.25 mg to about 2 mg) (e.g., daily).

In some embodiments, the methods include administering a dose of from about 50mg to about 1000 mg of a CAI (e.g., acetazolamide), from about 100 mg to about 800 mg of a CAI (e.g., acetazolamide), from about 250 mg to about 750 mg of a CAI (e.g., acetazolamide), from about 500 mg to about 750 mg of a CAI (e.g., acetazolamide), or from about 450 mg to about 650 mg of a CAI (e.g., acetazolamide). In some embodiments, the CAI is administered daily. In some embodiments, the CAI or a pharmaceutically acceptable salt thereof is administered daily before sleep time, e.g., immediately before sleep time or 15-60 minutes before sleep time.

In methods comprising administration of an MRA, such as oxybutynin or (R)-oxybutynin or a pharmaceutically acceptable salt thereof (or another MRA), the dose of oxybutynin or (R)-oxybutynin or pharmaceutically acceptable salt thereof may be from about 1 mg to about 25 mg (or a dose equivalent thereof of another MRA), or in some embodiments, from about 2 mg to about 15 mg. In some embodiments, the dose of oxybutynin or pharmaceutically acceptable salt thereof is from about 2.5 mg to about 10 mg, e.g., 5 mg. In some embodiments, the dose of (R)-oxybutynin or pharmaceutically acceptable salt thereof is from about 1 mg to about 5 mg, e.g., 2.5 mg. In some embodiments, the dose of oxybutynin or (R)-oxybutynin or pharmaceutically acceptable salt thereof is from about 1 mg to about 10 mg.

### Pharmaceutical Compositions

Also provided herein are pharmaceutical compositions comprising (i) a cannabinoid and (ii) a carbonic anhydrase inhibitor (CAI) selected from the group consisting of dichlorophenamide, zonisamide, ethoxzolamide, topiramate, and sultiame, or a pharmaceutically acceptable salt thereof, and one or more pharmaceutically acceptable carriers or excipients, wherein the compositions do not contain a norepinephrine reuptake inhibitor (NRI). Further provided herein are pharmaceutical compositions comprising a non-tetrahydrocannabinol (non-THC) cannabinoid and (ii) a carbonic anhydrase inhibitor (CAI), or a pharmaceutically acceptable salt thereof, and one or more pharmaceutically acceptable carriers or excipients, wherein the compositions do not contain a norepinephrine reuptake inhibitor (NRI). In some embodiments, the pharmaceutical compositions further comprise a muscarinic receptor antagonist (MRA). The cannabinoid, non-THC canabinoid, the CAI, and optionally the MRA are active pharmaceutical ingredients of the pharmaceutical compositions. The active pharmaceutical ingredients can be in a single composition or in separate compositions. In some embodiments, the pharmaceutical compositions do not contain a norepinephrine reuptake inhibitor (NRI).

Exemplary cannabinoids include cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA) and pharmaceutically acceptable salts thereof, or any combination thereof.

In some embodiments, the cannabinoid is CBD.

In some embodiments, the cannabinoid is THC. In some embodiments, the cannabinoid is synthetic THC. In some embodiments, the cannabinoid is a synthetic THC derivative (e.g., nabilone). In some embodiments, the cannabinoid is an enantiomerically pure form of THC (e.g., dronabinol). Dronabinol is synthetic delta-9-tetrahydrocannabinol (delta-9-THC).

Additional exemplary cannabinoids include commercially available cannabinoids such as Epidiolex® (CBD oral solution), Sativex® (nabiximols), Cesamet® (nabilone), Marinol® (dronabinol), and Acomplia® (rimonabant).

Additional exemplary cannabinoids include investigational cannabinoids such as SCI-110 (THX-110), AM-251, AM-630, HU-308, ABX-1431, RAD-011, Liquid Structure CBD, ART12.11 (CBD cocrystals), GWP-42006), CMX-020, ECP022A, Dronabinol buccal, Nabiolone controlled release, NE-1940, Olorinab, Drinabant, MDMB-FUBINACA, 5F-AB-PINACA, 5F-ADB, 5F-AMB, 5F-APINACA, AB-FUBINACA, AB-CHFUPYCA, AB-CHMINACA, AB-PINACA, ADB-CHMINACA, ADB-FUBINACA, ADSB-FUB-187,ADB-PINACA, ADBICA, APICA, Adamantyl-THPINACA, STS-135, AB-001, A-834,735, A-796,260, A-836,339, JWH-200, JWH-018, GUB-APINACA, APP-FUBINACA, MDMB-CHMICA, PX-1, PX-2, PX-3, CP-55,940, Dimethylheptylpyran, HU-210, HU-331, SR144528, WIN 55,212-2, Levonantradol, AM-2201, MEPIRAPIM, JWH-133 and Levonantradol.

Exemplary non-THC cannabinoids include cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), and cannabicyclol (CBL), or any combination thereof, or a pharmaceutically acceptable salt thereof.

In some embodiments, the non-THC cannabinoid is CBD, or a pharmaceutically acceptable salt thereof.

In some embodiments, the CBD, or a pharmaceutically acceptable salt thereof, is present in an amount of from about 0.5 to about 300 mg. In some embodiments, the CBD, or a pharmaceutically acceptable salt thereof, is present in an amount of from about 1 to about 100 mg. In some embodiments, the CBD, or a pharmaceutically acceptable salt thereof, is present in an amount of from about 1 mg to about 10 mg. In some embodiments, the CBD, or a pharmaceutically acceptable salt thereof, is present in an amount of from about 10 mg to about 100 mg.

In some embodiments, the THC, or a pharmaceutically acceptable salt thereof, is present in an amount of from about 0.1 to about 30 mg. In some embodiments, the THC, or a pharmaceutically acceptable salt thereof, is present in an amount of from about 1 to about 20 mg. In some embodiments, the THC, or a pharmaceutically acceptable salt thereof, is present in an amount of from about 0.5 to about 20 mg. In some embodiments, the THC, or a pharmaceutically acceptable salt thereof, is present in an amount of from about 0.25 to about 10 mg.

Exemplary CAIs include acetazolamide, dichlorophenamide, dorzolamide, brinzolamide, methazolamide, zonisamide, ethoxzolamide, topiramate, sultiame, and any combination thereof, including pharmaceutically acceptable salts thereof.

In some embodiments, the CAI is sultiame, or a pharmaceutically acceptable salt thereof. In some embodiments, the sultiame is present in an amount of from about 200 mg to about 400 mg.

Exemplary muscarinic receptor antagonists (MRAs) include atropine, propantheline, bethanechol, solifenacin, darifenacin, tolterodine, fesoterodine, trospium, and oxybutynin, and pharmaceutically acceptable salts thereof, which have activity on the M2 receptor. Other exemplary antimuscarinics include anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, and pharmaceutically acceptable salts thereof.

In some embodiments, the muscarinic receptor antagonist is oxybutynin or (R)-oxybutynin, or a pharmaceutically acceptable salt thereof. As used herein, (R)-oxybutynin refers to the (R)-oxybutynin stereoisomer substantially free of other stereoisomers of oxybutynin.

In some embodiments, the oxybutynin or a pharmaceutically acceptable salt thereof is present in the composition in an amount of from about 1 to about 15 mg. In some embodiments, the oxybutynin or a pharmaceutically acceptable salt thereof is present in the composition in an amount of from about 2 mg to about 10 mg. In some embodiments, the (R)-oxybutynin or a pharmaceutically acceptable salt thereof is present in the composition in an amount of from about 0.5 to about 10 mg. In some embodiments, the (R)-oxybutynin or a pharmaceutically acceptable salt thereof is present in the composition in an amount of from about 1 mg to about 5 mg. In some embodiments, the (i) cannabinoid, (ii) CAI, and, optionally the (iii) MRA are formulated in a single composition. In some embodiments, the (i) non-THC cannabinoid, (ii) CAI, and, optionally the (iii) MRA are formulated in a single composition.

As used herein the language “pharmaceutically acceptable carrier” includes saline, solvents, dispersion media, diluents, fillers, coatings, antibacterial and antifungal agents, isotonic and absorption delaying agents, and the like, compatible with pharmaceutical administration.

The active pharmaceutical ingredients (APIs) for use in the present invention may be provided as pharmaceutically acceptable salts. For example, in some embodiments, when MRAs are present, oxybutynin is oxybutynin chloride. In some embodiments, (R)-oxybutynin is (R)-oxybutynin chloride.

Pharmaceutical compositions are typically formulated to be compatible with its intended route of administration. Examples of routes of administration include systemic oral or transdermal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of the compounds as described herein can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

In some embodiments, the pharmaceutical compositions disclosed herein are for use in treating a condition associated with pharyngeal airway muscle collapse during sleep. In some embodiments, the condition associated with pharyngeal airway muscle collapse is sleep apnea (e.g., obstructive sleep apnea (OSA)) or snoring (e.g., simple snoring).

### Kits and Combinations

Also provided herein is a kit comprising (i) a cannabinoid and (ii) a carbonic anhydrase inhibitor (CAI) selected from the group consisting of dichlorophenamide, zonisamide, ethoxzolamide, topiramate, and sultiame, or a pharmaceutically acceptable salt thereof, and optionally (iii) a muscarinic receptor antagonist (MRA), wherein the kit does not contain a norepinephrine reuptake inhibitor (NRI). Further provided herein is a kit comprising (i) a non-THC cannabinoid and (ii) a carbonic anhydrase inhibitor (CAI) selected, or a pharmaceutically acceptable salt thereof, and optionally (iii) a muscarinic receptor antagonist (MRA), wherein the kit does not contain a norepinephrine reuptake inhibitor (NRI). For example, the kits may comprise separate pharmaceutical compositions with each composition lacking a norepinephrine reuptake inhibitor (NRI). The kits can be used for treating a subject having a condition associated with pharyngeal airway collapse. Various embodiments of kits will be apparent from the detailed description provided herein, including from the compositions and methods described herein.

Also provided herein is a cannabinoid and a carbonic anhydrase inhibitor (CAI) selected from the group consisting of dichlorophenamide, zonisamide, ethoxzolamide, topiramate, and sultiame, or a pharmaceutically acceptable salt thereof, and optionally a muscarinic receptor antagonist (MRA), for use in treating a subject having a condition associated with pharyngeal airway collapse, in the absence of a NRI therapy.

Also provided herein is a non-THC cannabinoid and a carbonic anhydrase inhibitor (CAI), or a pharmaceutically acceptable salt thereof, and optionally a muscarinic receptor antagonist (MRA), for use in treating a subject having a condition associated with pharyngeal airway collapse, in the absence of a NRI therapy. Further provided herein is a therapeutic combination of a cannabinoid and a carbonic anhydrase inhibitor (CAI) selected from the group consisting of dichlorophenamide, zonisamide, ethoxzolamide, topiramate, and sultiame, or a pharmaceutically acceptable salt thereof, and optionally a muscarinic receptor antagonist (MRA), for use in treating a subject having a condition associated with pharyngeal airway collapse, in the absence of a NRI therapy. Also provided herein is a therapeutic combination of a non-THC cannabinoid and a carbonic anhydrase inhibitor (CAI), or a pharmaceutically acceptable salt thereof, and optionally a muscarinic receptor antagonist (MRA), for use in treating a subject having a condition associated with pharyngeal airway collapse, in the absence of a NRI therapy. The therapeutic combinations may be used for treating a condition associated with pharyngeal airway collapse, such as sleep apnea or snoring.

Various embodiments of combinations and therapeutic combinations will be apparent from the detailed description provided herein, including from the compositions and methods described herein.

### EXAMPLES

The invention is further described in the following examples, which do not limit the scope of the invention described in the claims.

### Example 1. Crossover Study

A placebo-controlled, double-blinded, randomized, crossover trial in OSA human patients is performed. Participants receive treatment as follows: (1) once daily a cannabinoid (e.g., CBD or THC) plus a a carbonic anhydrase inhibitor (CAI) selected from the group consisting of dichlorophenamide, zonisamide, ethoxzolamide, topiramate, and sultiame or placebo; or (2) once daily a non-THC cannabinoid (e.g., CBD) plus a CAI or placebo in randomized order 30 minutes before sleep. The treatment is evaluated for its ability to reduce the apnea hypopnea index and improve OSA severity. Additional benefits evaluated may be increased genioglossus muscle responsiveness to an increase in ventilatory drive, improved upper airway muscle activity, improved ventilation, increased oxygen levels (SaO₂), increased total sleep time, improved hypoxic burden, and improved sleep efficiency.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with pharyngeal airway collapse, the method comprising administering to a subject in need thereof an effective amount of (i) a cannabinoid and (ii) a carbonic anhydrase inhibitor (CAI) selected from the group consisting of dichlorophenamide, zonisamide, ethoxzolamide, topiramate, and sultiame, or a pharmaceutically acceptable salt thereof, in the absence of a norepinephrine reuptake inhibitor (NRI) therapy.

2. The method of claim 1, wherein the (i) cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA), or any combination thereof, or a pharmaceutically acceptable salt thereof.

3. The method of claim 2, wherein the cannabinoid is CBD.

4. The method of claim 2, wherein the cannabinoid is THC.

5. The method of claim 1, wherein the (i) cannabinoid is dronabinol.

6. The method of any one of claims 1-5, wherein the CAI is sultiame, or a pharmaceutically acceptable salt thereof.

7. The method of claim 6, wherein the sultiame, or a pharmaceutically acceptable salt thereof, is administered at a dosage of about 200 to about 400 mg.

8. The method of claim 3, wherein CBD is administered at a dosage of from about 0.5 to about 300 mg.

9. The method of claim 4, wherein THC is administered at a dosage of from about 0.1 to about 30 mg.

10. The method of any one of claims 1-9, wherein the method further comprises administering (iii) a muscarinic receptor antagonist (MRA) to the subject.

11. The method of claim 10, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

12. The method of claim 10, wherein the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof.

13. The method of claim 11, wherein the MRA is oxybutynin or a pharmaceutically acceptable salt thereof.

14. The method of claim 11, wherein the MRA is (R)-oxybutynin or a pharmaceutically acceptable salt thereof.

15. The method of claim 13, wherein the oxybutynin or pharmaceutically acceptable salt thereof is administered at a dose of from about 1 to about 15 mg.

16. The method of claim 15, wherein the oxybutynin or pharmaceutically acceptable salt thereof is administered at a dose of from about 2 mg to about 10 mg.

17. The method of claim 14, wherein the (R)-oxybutynin or pharmaceutically acceptable salt thereof is administered at a dose of from about 0.5 to about 10 mg.

18. The method of claim 17, wherein the (R)-oxybutynin or pharmaceutically acceptable salt thereof is administered at a dose of from about 1 mg to about 5 mg.

19. The method of any one of claims 1-9, wherein the (i) cannabinoid and (ii) CAI are administered in single composition.

20. The method of any one of claims 10-19, wherein the (i) cannabinoid, (ii) CAI, and (iii) MRA are administered in single composition.

21. The method of claim 19 or 20, wherein the single composition is an oral administration form.

22. The method of claim 21, wherein the oral administration form is a syrup, pill, tablet, troche, capsule, or patch.

23. The method of any one of claims 1-22, wherein the condition associated with pharyngeal airway collapse is sleep apnea.

24. The method of claim 23, wherein the condition associated with pharyngeal airway collapse is obstructive sleep apnea (OSA).

25. The method of any one of claims 1-22, wherein the condition associated with pharyngeal airway collapse is snoring.

26. The method of claim 25, wherein the condition associated with pharyngeal airway collapse is simple snoring.

27. The method of any one of claims 1-26, wherein the subject is in a non-fully conscious state.

28. The method of claim 27, wherein the non-fully conscious state is sleep.

29. A method of treating a subject having a condition associated with pharyngeal airway collapse, the method comprising administering to a subject in need thereof an effective amount of (i) a non-tetrahydrocannabinol (non-THC) cannabinoid and (ii) a carbonic anhydrase inhibitor (CAI), or a pharmaceutically acceptable salt thereof, in the absence of a norepinephrine reuptake inhibitor (NRI) therapy.

30. The method of claim 29, wherein the (i) non-THC cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), and cannabicyclol (CBL), or any combination thereof, or a pharmaceutically acceptable salt thereof.

31. The method of claim 30, wherein the non-THC cannabinoid is CBD.

32. The method of claim 29, wherein the (ii) CAI is selected from the group consisting of acetazolamide, dichlorophenamide, dorzolamide, brinzolamide, methazolamide, zonisamide, ethoxzolamide, topiramate, sultiame, or a pharmaceutically acceptable salt thereof.

33. The method of claim 32, wherein the CAI is acetazolamide or a pharmaceutically acceptable salt thereof.

34. The method of any one of claims 29-33, wherein the CAI, such as acetazolamide, or a pharmaceutically acceptable salt thereof, is administered at a dosage of from about 250 mg to about 750 mg.

35. The method of claim 34, wherein the CAI, such as acetazolamide, or a pharmaceutically acceptable salt thereof, is administered at a dosage of about 500 mg.

36. The method of claim 31, wherein CBD is administered at a dosage of from about 0.5 to about 300 mg.

37. The method of any one of claims 29-36, wherein the method further comprises administering (iii) a muscarinic receptor antagonist (MRA) to the subject.

38. The method of claim 37, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

39. The method of claim 37, wherein the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof.

40. The method of claim 38, wherein the MRA is oxybutynin or a pharmaceutically acceptable salt thereof.

41. The method of claim 38, wherein the MRA is (R)-oxybutynin or a pharmaceutically acceptable salt thereof.

42. The method of claim 40, wherein the oxybutynin or pharmaceutically acceptable salt thereof is administered at a dose of from about 1 to about 15 mg.

43. The method of claim 42, wherein the oxybutynin or pharmaceutically acceptable salt thereof is administered at a dose of from about 2 mg to about 10 mg.

44. The method of claim 41, wherein the (R)-oxybutynin or pharmaceutically acceptable salt thereof is administered at a dose of from about 0.5 to about 10 mg.

45. The method of claim 44, wherein the (R)-oxybutynin or pharmaceutically acceptable salt thereof is administered at a dose of from about 1 mg to about 5 mg.

46. The method of any one of claims 29-36, wherein the (i) non-THC cannabinoid and (ii) CAI are administered in single composition.

47. The method of any one of claims 37-45, wherein the (i) non-THC cannabinoid, (ii) CAI, and (iii) MRA are administered in single composition.

48. The method of claim 46 or 47, wherein the single composition is an oral administration form.

49. The method of claim 48, wherein the oral administration form is a syrup, pill, tablet, troche, capsule, or patch.

50. The method of any one of claims 29-49, wherein the condition associated with pharyngeal airway collapse is sleep apnea.

51. The method of claim 50, wherein the condition associated with pharyngeal airway collapse is obstructive sleep apnea (OSA).

52. The method of any one of claims 29-49, wherein the condition associated with pharyngeal airway collapse is snoring.

53. The method of claim 52, wherein the condition associated with pharyngeal airway collapse is simple snoring.

54. The method of any one of claims 29-53, wherein the subject is in a non-fully conscious state.

55. The method of claim 54, wherein the non-fully conscious state is sleep.

56. A pharmaceutical composition comprising (i) a cannabinoid and (ii) a carbonic anhydrase inhibitor (CAI) selected from the group consisting of dichlorophenamide, zonisamide, ethoxzolamide, topiramate, and sultiame, or a pharmaceutically acceptable salt thereof, and one or more pharmaceutically acceptable carriers or excipients, wherein the composition does not contain a norepinephrine reuptake inhibitor (NRI).

57. The composition of claim 56, wherein the (i) cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA), or any combination thereof, or a pharmaceutically acceptable salt thereof.

58. The composition of claim 57, wherein the cannabinoid is CBD.

59. The composition of claim 57, wherein the cannabinoid is THC.

60. The composition of claim 56, wherein the (i) cannabinoid is dronabinol.

61. The composition of any one of claims 56-60, wherein the CAI is sultiame or a pharmaceutically acceptable salt thereof. 62 The composition of claim 61, wherein the sultiame, or a pharmaceutically acceptable salt thereof, is present in an amount of about 200 to about 400 mg.

63. The composition of claim 58, wherein CBD is present in an amount of from about 0.5 to about 300 mg.

64. The composition of claim 59, wherein THC is present in an amount of from about 0.1 to about 30 mg.

65. A pharmaceutical composition comprising (i) a non-tetrahydrocannabinol (non-THC) cannabinoid and (ii) a carbonic anhydrase inhibitor (CAI), or a pharmaceutically acceptable salt thereof, and one or more pharmaceutically acceptable carriers or excipients, wherein the composition does not contain a norepinephrine reuptake inhibitor (NRI).

66. The composition of claim 65, wherein the (i) non-THC cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), and cannabicyclol (CBL), or any combination thereof, or a pharmaceutically acceptable salt thereof.

67. The composition of claim 66, wherein the non-THC cannabinoid is CBD.

68. The composition of claim 65, wherien the (ii) CAI is selected from the group consisting of acetazolamide, dichlorophenamide, dorzolamide, brinzolamide, methazolamide, zonisamide, ethoxzolamide, topiramate, sultiame, or a pharmaceutically acceptable salt thereof.

69. The composition of claim 68, wherein the CAI is acetazolamide or a pharmaceutically acceptable salt thereof.

70. The composition of any one of claims 65-69, wherein the CAI, such as acetazolamide, or a pharmaceutically acceptable salt thereof, is present in an amount of from about 250 mg to about 750 mg.

71. The composition of claim 70, wherein the CAI, such as acetazolamide, or a pharmaceutically acceptable salt thereof, is present in an amount of about 500 mg.

72. The composition of claim 67, wherein CBD is present in an amount of from about 0.5 to about 300 mg.

73. The composition of any one of claims 56-72, wherein the composition further comprises (iii) a muscarinic receptor antagonist (MRA).

74. The composition of claim 73, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

75. The composition of claim 73, wherein the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof.

76. The composition of claim 74, wherein the MRA is oxybutynin or a pharmaceutically acceptable salt thereof.

77. The composition of claim 74, wherein the MRA is (R)-oxybutynin or a pharmaceutically acceptable salt thereof.

78. The composition of claim 76, wherein the oxybutynin or a pharmaceutically acceptable salt thereof is present in an amount of from about 1 to about 15 mg.

79. The composition of claim 76, wherein the oxybutynin or a pharmaceutically acceptable salt thereof is present in an amount of from about 2 mg to about 10 mg.

80. The composition of claim 77, wherein the (R)-oxybutynin or a pharmaceutically acceptable salt thereof is present in an amount of from about 0.5 to about 10 mg.

81. The composition of claim 77, wherein the (R)-oxybutynin or a pharmaceutically acceptable salt thereof is present in an amount of from about 1 mg to about 5 mg.

82. The composition of any one of claims 56-64, wherein the (i) cannabinoid, (ii) CAI, and, optionally the (iii) MRA, are formulated in a single composition.

83. The composition of any one of claims 65-81, wherein the (i) non-THC cannabinoid, (ii) CAI, and, optionally the (iii) MRA, are formulated in a single composition.

84. The composition of claim 82 or 83, wherein the single composition is an oral administration form.

85. The composition of claim 84, wherein the oral administration form is a syrup, pill, tablet, troche, capsule, or patch.

86. The composition of any one of claims 56-85, for use in treating a subject having a condition associated with pharyngeal airway collapse.

87. The composition of claim 86, wherein the condition associated with pharyngeal airway collapse is sleep apnea.

88. The composition of claim 86, wherein the condition associated with pharyngeal airway collapse is obstructive sleep apnea (OSA).

89. The composition of claim 86, wherein the condition associated with pharyngeal airway collapse is snoring.

90. The composition of claim 89, wherein the condition associated with pharyngeal airway collapse is simple snoring.

91. The composition of any one of claims 86-90, wherein the subject is in a non-fully conscious state.

92. The composition of claim 91, wherein the non-fully conscious state is sleep.

93. A kit comprising (i) a cannabinoid and (ii) a carbonic anhydrase inhibitor (CAI) selected from the group consisting of dichlorophenamide, zonisamide, ethoxzolamide, topiramate, and sultiame, or a pharmaceutically acceptable salt thereof, and optionally (iii) a muscarinic receptor antagonist (MRA), wherein the kit does not contain a norepinephrine reuptake inhibitor (NRI).

94. A kit comprising (i) a non-THC cannabinoid and (ii) a carbonic anhydrase inhibitor (CAI) selected, or a pharmaceutically acceptable salt thereof, and optionally (iii) a muscarinic receptor antagonist (MRA), wherein the kit does not contain a norepinephrine reuptake inhibitor (NRI).

95. The kit of claim 93 or 94, for use in treating a subject having a condition associated with pharyngeal airway collapse.

96. A cannabinoid and a carbonic anhydrase inhibitor (CAI) selected from the group consisting of dichlorophenamide, zonisamide, ethoxzolamide, topiramate, and sultiame, or a pharmaceutically acceptable salt thereof, and optionally a muscarinic receptor antagonist (MRA), for use in treating a subject having a condition associated with pharyngeal airway collapse in the absence of a norepinephrine reuptake inhibitor (NRI) therapy.

97. A non-THC cannabinoid and a carbonic anhydrase inhibitor (CAI), or a pharmaceutically acceptable salt thereof, and optionally a muscarinic receptor antagonist (MRA), for use in treating a subject having a condition associated with pharyngeal airway collapse in the absence of a norepinephrine reuptake inhibitor (NRI) therapy.
